(19) United States
(12) Patent Application Publication
(10) Pub. No.: US 2023/0100385 A1
(43) Pub. Date: Mar. 30, 2023

(54) Pharmaceutical Composition Comprising Cannabinoid

(71) Applicant: Blackhawk Partners Ltd., Limassol OT (CY)

(72) Inventor: Peter Whitton, Leicester (GB)

(21) Appl. No.: 18/076,147

(22) Filed: Dec. 6, 2022

Related U.S. Application Data
(62) Division of application No. 16/816,631, filed on Mar. 12, 2020.

(30) Foreign Application Priority Data
Oct. 11, 2019 (GB) 1914719.8

(51) Int. Cl. A61K 36/185; A61K 31/05; A61K 38/06; A61K 31/352

(52) U.S. Cl. CPC A61K 36/185; A61K 31/05; A61K 38/063; A61K 31/352; A61K 2236/17; A61K 2236/33; A61K 2236/51

(57) ABSTRACT

The present disclosure provides a pharmaceutical composition comprising a first pharmaceutically active portion comprising one or more cannabinoids extracted from Cannabis plant material; a second pharmaceutically active portion comprising glutathione extracted from Cannabis plant material and optionally one or more excipients; wherein the molar ratio of the second pharmaceutically active portion to the first pharmaceutically active portion is at least 0.5:1. The present disclosure also provides a method of manufacturing a pharmaceutical composition comprising the steps of: extracting a first fraction containing one or more cannabinoids from a Cannabis plant material using a polar solvent; extracting a second fraction containing from the glutathione from the same Cannabis plant material using water; and drying and combining the first fraction and the second fraction to form the pharmaceutical composition.

### FIELD OF THE INVENTION

The present invention relates to the pharmaceutical use of cannabinoids extracted from Cannabis plants and Cannabis plant cells.

### BACKGROUND

Historically, some forms of Cannabis sativa (Cannabis) have been used as illegal drugs th consumed for recreational purposes. This is primarily due to the effect of the psychoactive compound tetrahydrocannabinol (THC) a cannabinoid that is present in Cannabis. However, Cannabis also contains a large number of other cannabinoids including, but not limited to, cannabidiol, and cannabinol.

Cannabis has been smoked but it has also been ingested either in plant form or as a resin. More recently Cannabis has been taken for therapeutic reasons to treat a variety of conditions with varying degrees of success. Due to the illegal nature of Cannabis, even such therapeutic uses of the drug have typically still involved smoking or ingesting the whole plant or resin and there has been little research into the efficacy of such therapeutic uses. Further, due to Cannabis being illegal, little or no research was carried out into potential toxic or beneficial effects of smoking or ingestion of Cannabis.

More recently, Cannabis has been either decriminalised or legalised in many jurisdictions and, as a result, the potential therapeutic benefits of the whole plant and/or the chemical components have begun to be investigated. Further, there have been efforts to extract and isolate the various cannabinoids and to manufacture pharmaceutical compositions utilising the isolated cannabinoids. This has the advantage that relevant cannabinoids can then be provided in an easily ingestable form independent of the other cannabinoids and, in particular, without the psychoactive effects of the THC. However, as isolated cannabinoids have begun to be used in pharmaceutical compositions it has been discovered that they can have a hepatoxic effect.

Examination of the molecular formulae of the principle cannabinoids show that they are most likely to be metabolised via the cytochrome p450 enzyme pathway in the liver. This involves the liver enzymes reacting the cannabinoids with glutathione within the liver to open the cannabinoids' ring structures and make them water soluble. The resulting water soluble compounds are then absorbed into the bloodstream and can cross the blood/brain barrier.

In individuals who have depleted glutathione stores in the liver or those who are on additional medication there may be insufficient glutathione within the liver to effectively metabolise the cannabinoids. This can lead to the total depletion of glutathione stores within the liver and the enzymatic detoxification pathways are likely to become saturated, leading to hepatotoxic side-effects. Over a period of time, if left untreated, this can lead to potentially fatal liver failure. Hepatoxicity such as this can be treated by administration of additional glutathione or cysteine (which may be given as acetylcysteine), which is the active moiety of the glutathione molecule. However, such treatment is time critical and the treatment has to be administered before any effects on the liver become irreversible. This is problematic as a patient may not experience any adverse side-effects until the liver damage is irreversible.

Glutathione occurs in most cells of the body in adequate amounts but some individuals have a deficiency. Such individuals are more likely to suffer hepatotoxic effects when taking cannabinoid based medication.

One current solution to this is to either use intact parts of a Cannabis plant or complete Cannabis sativa (Cannabis) cells rather than isolated cannabinoids. For example Cannabis cells produced in a cell culture environment can be used in place of isolated cannabinoids. Complete plant cells, whether from a plant source or from a cell culture environment, have all of the components of the Cannabis plant. In particular, Cannabis plants and their cells contain naturally high levels of glutathione. Therefore, ingesting complete plant cells provides a source of glutathione alongside the cannabinoids, which negates the hepatoxic effect of the cannabinoids. In this manner hepatoxicity has previously been avoided through the use of complete Cannabis cells, albeit generally unwittingly. It is to be noted that it because Cannabis contains glutathione that the potential hepatoxic effects of cannabinoids were not discovered or investigated until recently. The hepatoxic effects of cannabinoids has only been discovered when the cannabinoids have been separated from the Cannabis plant and used in pharmaceutical compositions.

However, although complete plant cells are useful in a variety of circumstances, in some cases it may not be preferable to form pharmaceutical compositions from complete plant cells. It may be desirable to have a pharmaceutical composition comprising one or more cannabinoids provided separate from a plant cell, for example in order to provide precise dosing of the relevant cannabinoid(s). In light of this, there is a need for improved pharmaceutical compositions comprising one or more cannabinoids in which there is no hepatoxicity.

### SUMMARY

The present invention provides a pharmaceutical composition consisting of:

a first pharmaceutically active portion consisting one or more cannabinoids;
a second pharmaceutically active portion consisting of one or more of glutathione, cysteine, acetylcysteine, alliin, bucillamine, carbocysteine, djenkolic acid, felinine, lanthionine, mecysteine hydrochloride, penicillamine cysteine disulphide, or any functional equivalent thereof; and
optionally one or more excipients; wherein
the molar ratio of the second pharmaceutically active portion to the first pharmaceutically active portion is at least 0.5:1.

As described herein, “cannabinoids” refers to the family of natural products that are present in Cannabis sativa, such as hemp, and that usually contain a 1,1′-di-menthyl-pyrange ring, a variedly derivatized aromatic ring, and a variedly unsaturated cyclohexyl ring and their immediate chemical precursors. The main cannabinoids are shown below.

It has been discovered that providing the pharmaceutical composition of the present invention can mitigate the hepatoxic effects of providing isolated cannabinoid(s). In particular, by providing a suitable mitigating compound along with the isolated cannabinoid(s) in a molar ratio of at least 0.5:1 hepatoxicity can be prevented. Suitable mitigating compounds include glutathione, cysteine, acetylcysteine, alliin, bucillamine, carbocysteine, djenkolic acid, felinine, lanthionine, mecysteine hydrochloride, penicillamine cysteine disulphide, or any functional equivalent thereof. Specifically, a compound that is functionally equivalent to the listed compounds is one that has an available S—H bond that can react with a double bond on an aromatic ring structure of a cannabinoid to open the cannabinoids' ring structures and make them water soluble. These mitigating compounds allow the cannabinoid(s) to be metabolised via the cytochrome p450 enzyme pathway in the liver. This involves the liver enzymes reacting the cannabinoids with the mitigating compound and/or a user's own glutathione store within the liver to open the cannabinoids' ring structures and make them water soluble.

Generally, at least one molecule of mitigating compound is required in the liver to metabolise each molecule of cannabinoid in the pharmaceutical composition when the composition is ingested. That is, in order to avoid hepatoxicity in a user there must be a molar ratio of mitigating compound to cannabinoid in the liver of at least 1:1. However, in healthy patients the liver will already have a supply of glutathione (which is a mitigating compound) such that it is not necessarily essential that the pharmaceutical composition of the present invention has a molar ratio of the second pharmaceutically active portion to the first pharmaceutically active portion of at least 1:1. Rather, molar ratios of 0.5:1, 0.6:1, 0.7:1, 0.8:1, or 0.9:1 may be sufficient depending on the specific patient and their glutathione stores, the amount of the first pharmaceutically active portion in the pharmaceutical composition, and the dosage frequency. However, in order to be completely safe it may be generally preferable that the second pharmaceutically active portion is provided in a molar ratio of at least 1:1 with the first pharmaceutically active portion.

The main cannabinoids are:

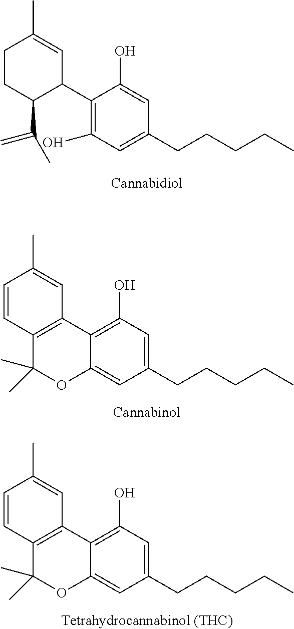

A pharmaceutical composition according to the present invention may comprise one or more of these cannabinoids and/or any other cannabinoids as the first pharmaceutically active portion.

The cannabinoid(s) of the first pharmaceutically active portion may be extracted from a Cannabis sativa plant, such as hemp, or from cultured Cannabis sativa plant cells or from any other suitable source. Additionally or alternatively, the first pharmaceutically active portion may comprise one or more synthetic cannabinoids.

The cannabinoid(s) may be extracted from Cannabis plant or Cannabis plant cells in any suitable manner. For example, the cannabinoid(s) may be extracted using solvent extraction and/or steam distillation. The skilled person will be readily aware of methods by which cannabinoids can be extracted from Cannabis. For example, cannabinoids can be extracted using simple solvent extraction techniques using ethanol as the solvent.

Generally, glutathione is not extracted from Cannabis plant or Cannabis plant cells when the cannabinoid(s) are extracted using conventional methods. This is because, whilst glutathione is water soluble, cannabinoids are not water soluble but soluble in non-polar solvents such as ethanol or acetone. Glutathione is insoluble in ethanol or acetone solutions of concentrations above 50% and only slightly soluble at lower concentrations of these solvents. Cannabinoids are not water soluble as they are non-polar molecules and generally require solvent extraction using ethanol or acetone at a high concentration i.e. with little or no water content. As a result, if a single source is used for both glutathione and a cannabinoid then it is generally necessary to utilise two separate extraction techniques sequentially on said single source. Extraction from cannabinoid(s) from Cannabis plant or Cannabis plant cells generally do not result in the extraction of glutathione.

If the second pharmaceutically active portion of the present invention consists of or comprises glutathione then the glutathione may be extracted from the same source as the cannabinoid(s) of the first pharmaceutically active portion. The extraction of the glutathione from the source may be done in any suitable manner and may be carried out before, after, or concurrently with the extraction of the cannabinoid(s) by utilising a suitable method. If the extractions are carried out concurrently then a suitable extraction method would generally include separate processes for extracting the glutathione and the cannabinoid(s). The source may be a Cannabis plant or a part thereof (processed or unprocessed), cultured Cannabis plant cells, or any other suitable source.

A pharmaceutical composition according to the present invention may comprise any suitable excipient. This includes, but is not limited to, sweeteners, flavouring agents, preservatives, and pH adjusters. Suitable sweeteners include, but are not limited to, sucralose, aspartame, acesulfame k and equivalents. It is considered that the skilled person will be able to determine suitable excipients for any specific composition according to the present invention.

The present invention also provides method of manufacturing a pharmaceutical composition according to claim 5 from Cannabis plant material, comprising the steps of:
i) extracting a first fraction containing one or more cannabinoids from the Cannabis plant material using a solvent;
ii) extracting a second fraction containing from the glutathione from the same Cannabis plant material; and
iii) drying and combining the first fraction and the second fraction to form the pharmaceutical composition.

As set out above, generally when cannabinoids are extracted from Cannabis plant material using a polar solvent any glutathione present in the plant material is not extracted. Therefore, if it is desirable to also extract glutathione from the plant material it is necessary to additionally extract the glutathione using a separate process, typically using water as a solvent. Extraction of the glutathione can be done in any suitable manner. The amounts of the first fraction and the second fraction that are combined can be controlled such that a suitable ratio of glutathione and cannabinoid are present in the pharmaceutical composition.

It is considered that the skilled person will be aware of many suitable methods for extracting cannabinoids from Cannabis plant material using a solvent. Similarly, it is considered that the skilled person will be aware of many suitable methods for extracting glutathione from Cannabis plant material using water. The method of the present invention is intended to encompass all such methods.

The Cannabis plant material of the method of the present invention may comprise cultured plant cells and/or plant parts including, but not limited to, leaves and stems. The plant material may be processed or unprocessed prior to extraction of the first fraction and the second fraction. Suitable processing includes, but is not limited to, drying and powdering.

It is considered that the skilled person will be able to determine the cannabinoid content of the first fraction and the glutathione content of the second fraction without difficulty, either before or after the first fraction and the second fraction are dried. As such, the skilled person will be able to combine the first fraction and the second fraction in a proper ratio to arrive at a pharmaceutical composition according to the present invention without difficulty.

In order to form the pharmaceutical composition of the present invention the first fraction and the second fraction may be combined with one or more excipients as discussed above.

### EXAMPLE

The components of a composition according to the present invention can be extracted from a single Cannabis sativa source in the following manner:
i) Extraction of cannabinoids

Solvent extraction of cannabinoids from Cannabis plant and separating the specific cannabinoids is well known in the art. U.S. Pat. No. 6,403,126 discloses one such method and the method disclosed in that patent is suitable for use with the present invention. In particular, the method of U.S. Pat. No. 6,403,126 is suitable for use in extracting cannabinoids from Cannabis sativa.

In this method Cannabis powder is extracted with a solvent, for example ethanol, for a period for two to four hours. The solvent may then be evaporated to leave a residue. The extracted residue can then be passed over a chromatographic column arranged to fractionate at least one cannabinoid. The fractionated cannabinoid(s) can then be used to form a pharmaceutical composition.
ii) Extraction of glutathione

Materials: 10 g dried Cannabis sativa leaf
100 ml de-ionised water
250 ml erhlingmeyer flask
Magnetic stirrer and bar
Filter paper
Filter funnel
Evaporating dish
Drying oven

The Cannabis sativa powder and the de-ionised water are added to the Ehrlingmeyer flask. The stirrer bar is placed into the flask and the mixture is stirred at a speed of 30 rpm for two hours. Optionally, a buffer may be used to maintain the pH of the water to 7.5 pH.

After stirring, the mixture is poured through the filter paper into the evaporating dish. The evaporating dish is placed in the drying oven at a temperature of 106° C. until completely evaporated. The resulting residue is a mixture of water soluble plant compounds from the powdered Cannabis sativa and includes a high percentage of glutathione.

If necessary, the residue can be further purified by removing other dissolved compounds using ethanol as a solvent and then retaining the non-dissolved residue. The glutathione content of the residue may be assayed by high performance liquid chromatography.

It is noted that the glutathione extraction method set out above does not generally result in pure glutathione. Further processing is required to obtain pure glutathione. However, for the purposes of the present invention it is not necessarily essential that pure glutathione is utilised so long as the extract from the Cannabis contains sufficient glutathione to provide the appropriate ratio with the cannabinoid(s) it is not essential that the extract is pure glutathione. Any other components of the extract are natural components of the Cannabis plant and, as such, are unlikely to be harmful to a consumer. In alternative embodiments compositions according to the present invention in which pure components are advantageous pharmaceutically pure glutathione from alternative sources may be utilised or further processing can be used to obtain pharmaceutically pure glutathione.

Subsequent to extraction the cannabinoid(s) can be combined with the glutathione and any suitable excipients to form a pharmaceutical composition according to the present invention.

What is claimed is:

1. A pharmaceutical composition comprising:
a first pharmaceutically active portion comprising one or more cannabinoids extracted from Cannabis plant material;
a second pharmaceutically active portion comprising glutathione extracted from Cannabis plant material, wherein the molar ratio of the second pharmaceutically active portion to the first pharmaceutically active portion is at least 0.5:1.

2. A pharmaceutical composition according to claim 1, wherein the molar ratio of the second pharmaceutically active portion to the first pharmaceutically active portion is at least 0.8:1.

3. A pharmaceutical composition according to claim 1, wherein the molar ratio of the second pharmaceutically active portion to the first pharmaceutically active portion is at least 1:1.

4. A pharmaceutical composition according to claim 1, wherein the first pharmaceutically active portion comprises cannabidiol.

5. A pharmaceutical composition according to claim 1, wherein the first pharmaceutically active portion comprises cannabinol.

6. A method of manufacturing a pharmaceutical composition according to claim 1 from Cannabis plant material, comprising the steps of:
extracting a first fraction comprising the one or more cannabinoids from the Cannabis plant material using a polar solvent;
extracting a second fraction comprising the glutathione from the Cannabis plant material using water;
drying the first fraction;
drying the second fraction; and,
combining the first fraction and the second fraction to form the pharmaceutical composition.

7. A method according to claim 6, wherein the polar solvent is ethanol.

8. A method according to claim 6, wherein the Cannabis plant material consists of Cannabis plant cells.

9. A method according to claim 6, wherein the Cannabis plant material comprises dried Cannabis plant.

10. A method according to claim 6, wherein the first fraction and the second fraction are combined with one or more excipients to form the pharmaceutical composition.

11. A pharmaceutical composition according to claim 1, wherein the first pharmaceutically active portion comprises tetrahydrocannabionol.
